(19) United States
(12) Patent Application Publication
(10) Pub. No.: US 2023/0201555 A1
(43) Pub. Date: Jun. 29, 2023

(54) HEMOSTATIC DEVICE

(71) Applicant: TERUMO KABUSHIKI KAISHA, Tokyo (JP)

(72) Inventors: Mizuki KABE, Yamaguchi (JP); Yuna HIDAKA, Ibaraki (JP)

(73) Assignee: TERUMO KABUSHIKI KAISHA, Tokyo (JP)

(21) Appl. No.: 18/170,798

(22) Filed: Feb. 17, 2023

Related U.S. Application Data
(63) Continuation of application No. PCT/JP2021/030675, filed on Aug. 20, 2021.

(30) Foreign Application Priority Data
Aug. 21, 2020 (JP) 2020-139907

(51) Int. Cl. A61M 39/06

(52) U.S. Cl. CPC A61M 39/06; A61M 2039/0673

(57) ABSTRACT

A hemostatic device is disclosed that includes a pressing member including a pressing portion configured to compress a first puncture site formed in a patient and a support member configured to fix the pressing portion. The support member has a first region where the pressing portion is disposed and a second region positioned outside the first region and configured such that a first band body, a second band body, and a third band body are connectable. At least two of a plurality of hole portions provided in the second region, such that any of the first band body, the second band body, and the third band body connected to the hole portions can be disposed so as to radially change in angle about the pressing portion positioned in the first region, are larger than the widths of the band bodies and configured in a non-linear shape.

### CROSS-REFERENCES TO RELATED APPLICATIONS

This application is a continuation of International Application No. PCT/JP2021/030675 filed on Aug. 20, 2021, which claims priority to Japanese Application No. 2020-139907 filed on Aug. 21, 2020, the entire content of both of which is incorporated herein by reference.

### TECHNOLOGICAL FIELD

The present disclosure generally relates to a hemostatic device.

### BACKGROUND DISCUSSION

In catheter procedures, lesion site handling or treatment is performed by introducing various medical elongated bodies into a blood vessel via a puncture site formed by puncturing the blood vessel in a patient's arm or hand. For example, U.S. Patent Application Publication No. 2019/0133602 discloses a hemostatic device for hemostasis at a puncture site formed in order to enable access to a hand-running blood vessel (including a distal radial artery).

The hemostatic device of U.S. Patent Application Publication No. 2019/0133602 includes a pressing member provided with a balloon applying a compressive force to a puncture site formed in a patient's hand and a plurality of band bodies for fixing the pressing member to the patient's hand. In addition, the plurality of band bodies include a winding band body disposed so as to be wound along the outer circumference of the hand and a finger hook band body disposed on an interdigital portion positioned between adjacent fingers.

In performing hemostasis at a puncture site in a patient's hand using the hemostatic device of U.S. Patent Application Publication No. 2019/0133602, an operator such as a doctor (hereinafter, referred to as “operator”) winds the winding band body along the outer circumference of the hand and, further, disposes a finger hook band body on the interdigital portion between the thumb and the index finger. With the pressing member disposed at and around the puncture site in the patient's hand, the operator can help prevent a positional deviation of the pressing member from the puncture site in the patient's hand by fixing the hemostatic device using each band body.

However, the hemostatic device described in U.S. Patent Application Publication No. 2019/0133602 may have the following problems.

The position of a hand-running blood vessel is different from patient to patient and patients have different physiques. As a result, an operator in procedures may form puncture sites at different positions in a patient's hand. For example, the operator may form a puncture site in the anatomical snuff box positioned on the dorsal side of the patient's hand or may form a puncture site at a position on the side peripheral to the snuff box (side closer to the fingertip than the snuff box).

The hemostatic device of U.S. Patent Application Publication No. 2019/0133602 is not intended to be used for each of puncture sites formed at different positions in one hand. Accordingly, the position of connection between the winding band body and the pressing member is fixed. Accordingly, the hemostatic device of U.S. Patent Application Publication No. 2019/0133602 has a relatively low degree of freedom in the hand mounting position of the winding band body.

In a case where the hemostatic device of U.S. Patent Application Publication No. 2019/0133602 is used for hemostasis at puncture sites at different positions in one hand, it is considered possible to dispose the winding band body at an appropriate patient hand position so as not to interfere with, for example, the patient's finger movement when hemostasis is performed at one of the puncture sites (for example, the puncture site in the snuff box). Meanwhile, in a case where the hemostatic device of U.S. Patent Application Publication No. 2019/0133602 is used for hemostasis at another puncture site (for example, a puncture site positioned peripheral to the snuff box), the winding band body is wound at a position where mounting is not assumed originally (for example, a position closer to the peripheral side of the hand than the snuff box). The winding band body restrains the movement of the hand in a case where the winding band body is wound at the position closer to the peripheral side of the hand than the snuff box. The winding band body deviates from the hand in the event of an operation such as the patient spreading the hand with the winding band body mounted on the hand as described above. As a result of the deviation of the winding band body from the hand, the pressing member connected to the winding band body deviates from the puncture site in the hand. Accordingly, it can be difficult to appropriately and stably perform hemostasis at a puncture site in a hand with the hemostatic device of U.S. Patent Application Publication No. 2019/0133602.

### SUMMARY

A hemostatic device is disclosed that is capable of preventing a patient's hand movement from being restrained with a pressing member disposed at a puncture site in the patient's hand and can be rather easily mounted onto the patient's hand.

A hemostatic device according to the disclosure includes: a pressing member configured to compress a puncture site formed in a patient; a first band body configured to be connectable to the pressing member; a second band body configured to be connectable to the pressing member; and a third band body configured to be connectable to the pressing member, in which the pressing member has a pressing portion configured to compress the puncture site and a support member configured to fix the pressing portion, the support member has a first region where the pressing portion is disposed and a second region positioned outside the first region and configured such that the first band body, the second band body, and the third band body are connectable, the first band body, the second band body, and the third band body are configured to be respectively connectable to a plurality of hole portions provided in the second region, and at least two of the plurality of hole portions, such that any of the first band body, the second band body, and the third band body connected to the hole portions can be disposed so as to radially change in angle about the pressing portion in the first region, are larger than widths of the band bodies and configured in a non-linear shape.

According to the hemostatic device of the disclosure, at least two of the plurality of hole portions are larger than the widths of the band bodies and configured in a non-linear shape such that the band bodies connected to the hole portions can be disposed so as to radially change in angle about the pressing portion in the first region. Accordingly, in the hemostatic device, it is possible to adjust the angles and positions of two band bodies with respect to the patient's hand by moving the band bodies respectively connected to the two hole portions about the pressing portion so as to be radially disposed along the hole portions while disposing the pressing member at the puncture site formed in the patient's hand. As a result, with the hemostatic device, the two band bodies can be disposed on the patient's hand such that the movement of the patient's hand is not restrained, and the hemostatic device can be easily mounted onto the patient's hand.

According to an embodiment, a hemostatic device is disclosed which includes: a pressing member configured to compress a puncture site formed in a patient; a first band body configured to be connectable to the pressing member; a second band body configured to be connectable to the pressing member; a third band body configured to be connectable to the pressing member; the pressing member including a pressing portion configured to compress the puncture site and a support member configured to fix the pressing portion; the support member including a first region where the pressing portion is disposed and a second region positioned outside the first region; the first band body, the second band body, and the third band body are configured to be respectively connectable to a plurality of hole portions provided in the second region of the support member; and at least two of the plurality of hole portions having widths that are larger than a width of the first band body, a width of the second band body, and a width of the third band body, the at least two of the plurality of hole portions being configured in a non-linear shape, and wherein one or more of the first band body, the second band body, and the third band body are configured to be connected to the at least two hole portions and configured to radially change in angle about the pressing portion relative to the first region.

According to another embodiment, a hemostatic device is disclosed, which includes: an inflatable member configured to receive a fluid; a support member connected to the inflatable member, the support member including a first region where the inflatable member is disposed and a second region positioned outside the first region; a first band body configured to be connectable to the pressing member; a second band body configured to be connectable to the pressing member; a third band body configured to be connectable to the pressing member; the first band body, the second band body, and the third band body are configured to be respectively connectable to a plurality of hole portions provided in the second region of the support member; and wherein one or more of the first band body, the second band body, and the third band body are configured to be connected to the at least two hole portions and configured to radially change in angle about the inflatable portion relative to the first region.

According to an embodiment, a method is disclosed, which includes: wrapping a first band body, a second band body, and a third band body of a hemostatic device along an outer circumference of a puncture site of a living body, the hemostatic device includes a pressing member, the pressing member including a pressing portion configured to compress the puncture site of the living body and a support member configured to fix the pressing portion, the support member including a first region where the pressing portion is disposed and a second region positioned outside the first region, the first band body, the second band body, and the third band body being connected to a plurality of hole portions provided in the second region of the support member, and at least two of the plurality of hole portions having widths that are larger than a width of the first band body, a width of the second band body, and a width of the third band body; radially changing in angle about the pressing portion relative to the first region, one or more of the first band body, the second band body, and the third band body connected to the at least two hole portions; and injecting a fluid into of the pressing portion disposed on a surface positioned on a side opposite to a surface of the support member of the pressing member to apply a compressive force to the puncture site of the living body.

### BRIEF DESCRIPTION OF THE DRAWINGS

FIG. 1 is a diagram illustrating a hemostatic device according to an embodiment and is a plan view seen from the outer surface side of each band body.

FIG. 2 is a diagram illustrating the hemostatic device according to the embodiment and is a plan view seen from the inner surface side of each band body.

FIG. 3 is an enlarged plan view illustrating a part of the hemostatic device seen from the outer surface side of each band body.

FIG. 4 is an enlarged plan view illustrating a part of the hemostatic device seen from the inner surface side of each band body.

FIG. 5 is an enlarged plan view illustrating a part of the hemostatic device seen from the inner surface side of each band body.

FIG. 6 is a cross-sectional view of the hemostatic device along the arrows 6A-6A illustrated in FIG. 5, illustrating a state where an inflatable member is inflated.

FIG. 7 is a cross-sectional view of the hemostatic device along the arrows 7A-7A illustrated in FIG. 5, illustrating a state where the inflatable member is inflated.

FIG. 8 is a plan view of the hemostatic device seen from the outer surface side of each band body, illustrating states before and after sliding the second and third band bodies connected to a pressing member.

FIG. 9 is a perspective view illustrating a support member of the pressing member.

FIG. 10 is a perspective view illustrating the support member of the pressing member.

FIG. 11 is a diagram illustrating a patient's hand (right hand) for which the hemostatic device is used.

FIG. 12 is a diagram briefly illustrating a first example of use of the hemostatic device.

FIG. 13 is a diagram briefly illustrating the first example of use of the hemostatic device.

FIG. 14 is a diagram briefly illustrating the first example of use of the hemostatic device.

FIG. 15 is a partial cross-sectional view taken along the arrows 15A-15A illustrated in FIG. 14.

FIG. 16 is a partial cross-sectional view taken along the arrows 16A-16A illustrated in FIG. 14.

FIG. 17 is a diagram briefly illustrating a second example of use of the hemostatic device.

FIG. 18 is a diagram illustrating a patient's hand (left hand) for which the hemostatic device is used.

FIG. 19 is a diagram briefly illustrating a third example of use of the hemostatic device.

FIG. 20 is a diagram briefly illustrating a fourth example of use of the hemostatic device.

FIG. 21 is an enlarged plan view illustrating a part of a hemostatic device according to Modification Example 1.

FIG. 22 is a diagram illustrating a patient's hand (right hand) for which the hemostatic device is used.

FIG. 23 is a plan view briefly illustrating an example of use of the hemostatic device according to Modification Example 1.

FIG. 24 is a plan view briefly illustrating an example of use of the hemostatic device according to Modification Example 1.

### DETAILED DESCRIPTION

Set forth below with reference to the accompanying drawings is a detailed description of embodiments of a hemostatic device. Note that since embodiments described below are preferred specific examples of the present disclosure, although various technically preferable limitations are given, the scope of the present disclosure is not limited to the embodiments unless otherwise specified in the following descriptions. In the drawings, the same or corresponding parts are denoted by the same reference numerals. In the description of the present embodiment, the description of the same or corresponding parts will be omitted or simplified as appropriate. In addition, the dimensional ratios in the drawings are exaggerated for convenience of description and may differ from the actual ratios.

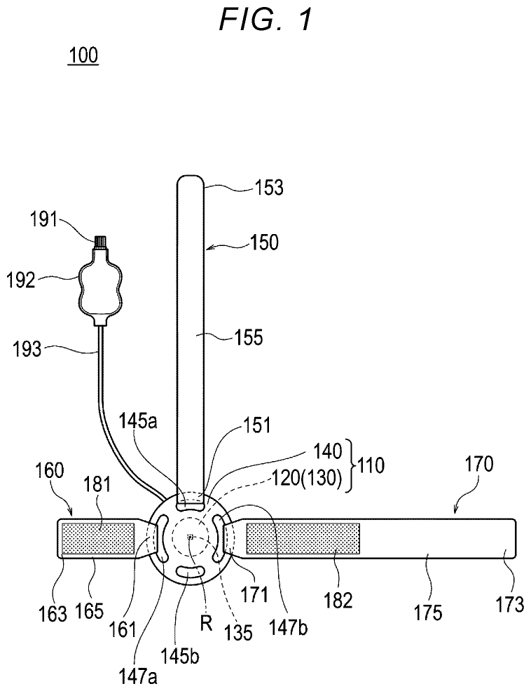

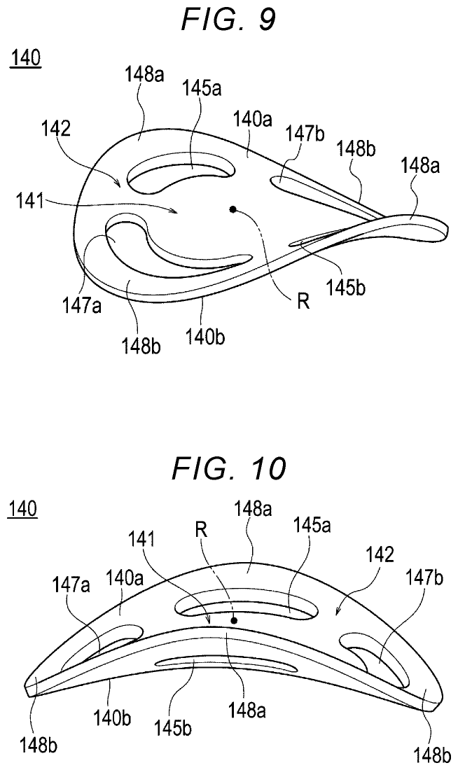

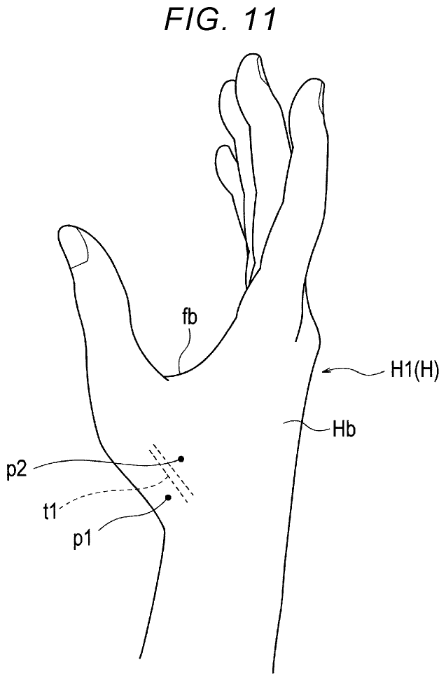

FIGS. 1 to 10 are diagrams for describing a hemostatic device 100 according to the present embodiment. FIGS. 11 to 20 are diagrams for describing examples of use of the hemostatic device 100.

As illustrated in, for example, FIGS. 11 and 14 to 16, the hemostatic device 100 can be used in removing the sheath tube of an introducer 200 placed at a puncture site (for example, each of puncture sites p1, p2, p3, and p4 to be described later) formed in a hand H positioned closer to the distal side (finger side) than a patient's forearm A so that hemostasis is performed at the puncture site.

Although the specific position of the puncture site that is subject to hemostasis by the hemostatic device 100 is not particularly limited, the following first puncture site p1, second puncture site p2, third puncture site p3, and fourth puncture site p4 are exemplified in the present embodiment. Note that in the present specification, the structure of each portion of the hemostatic device 100 will be described mainly through an example in which the hemostatic device 100 is used for hemostasis at the first puncture site p1.

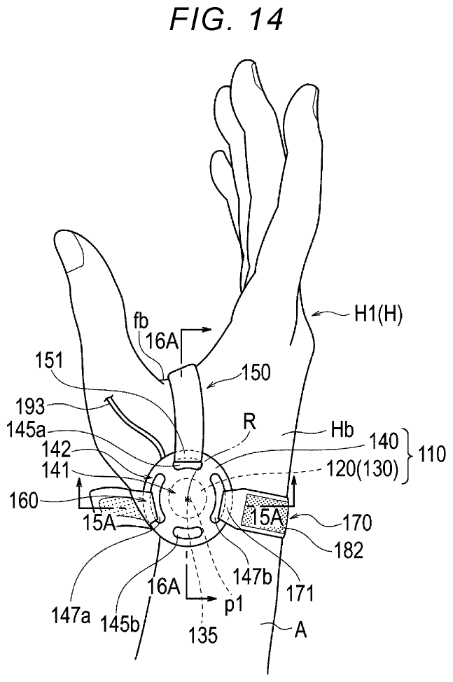

As illustrated in FIGS. 11 and 14, the first puncture site p1 is a puncture site formed in an artery B (hereafter, also referred to as “blood vessel B”) positioned in the snuff box of the palmar artery running on a back Hb side of a right hand H1 (hand H) positioned distal to the patient's forearm A. Note that the snuff box is the cavity of the hand positioned near the radius when the patient spreads the thumb of the hand H.

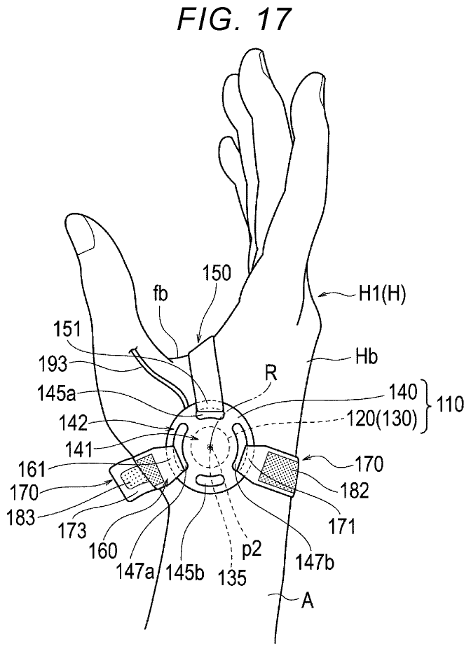

As illustrated in FIGS. 11 and 17, the second puncture site p2 is a puncture site formed in the distal radial artery positioned distal to the snuff box of the palmar artery running on the back Hb side of the patient's right hand H1. The second puncture site p2 is positioned closer to the distal side of the right hand H1 than the first puncture site p1 with respect to an extensor pollicis longus tendon t1 positioned in the back Hb of the patient's right hand H1.

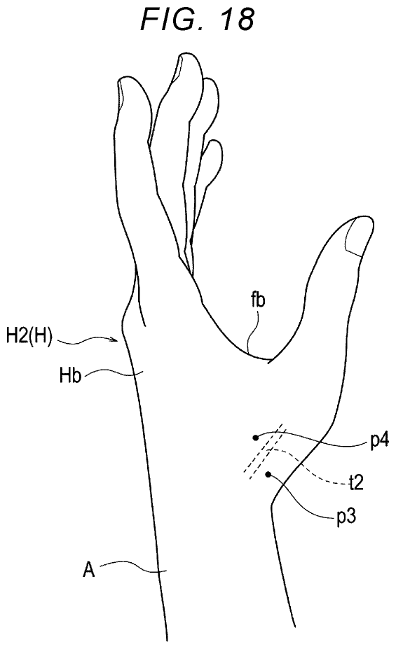

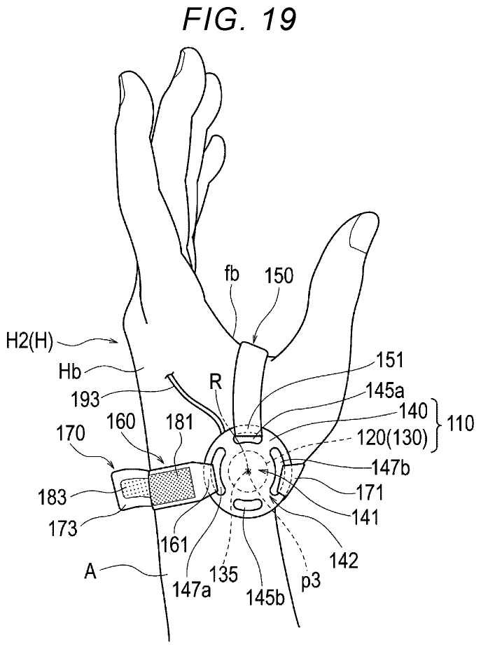

As illustrated in FIGS. 18 and 19, the third puncture site p3 is a puncture site formed in the artery positioned in the snuff box of the palmar artery running on the back Hb side of the patient's left hand H2 (hand H).

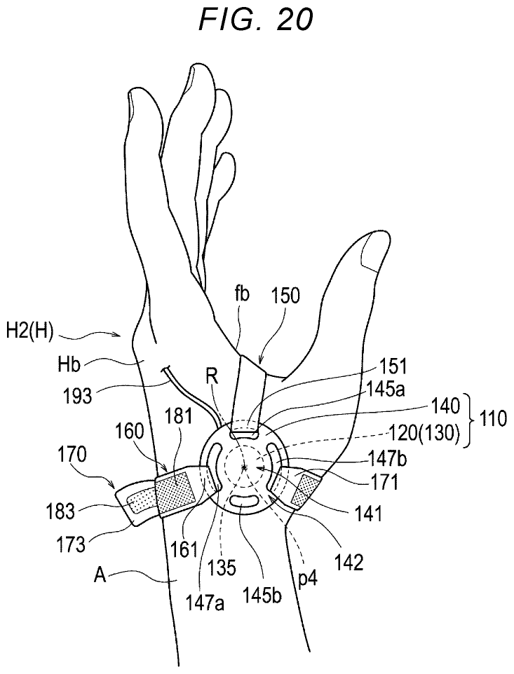

As illustrated in FIGS. 18 and 20, the fourth puncture site p4 is a puncture site formed in the distal radial artery positioned distal to the snuff box of the palmar artery running on the back Hb side of the patient's left hand H2. The fourth puncture site p4 is positioned closer to the distal side of the left hand H2 than the third puncture site p3 with respect to an extensor pollicis longus tendon t2 positioned in the back Hb of the patient's left hand H2.

Hereinafter, the hemostatic device 100 will be described in detail.

Generally speaking, as illustrated in FIGS. 1, 2, 14, 15, and 16, the hemostatic device 100 includes a pressing member 110 configured to compress the first puncture site p1 formed in the patient's right hand H1, a first band body 150 configured to be connectable to the pressing member 110, a second band body 160 configured to be connectable to the pressing member 110, and a third band body 170 configured to be connectable to the pressing member 110. As illustrated in FIGS. 1, 2, 14, 15, and 16, the first band body 150, the second band body 160, and the third band body 170 are connected to the pressing member 110. Specifically, the first band body 150, the second band body 160, and the third band body 170 are connected to the pressing member 110 via a plurality of hole portions provided in a second region 142 of a support member 140 (first hole portion 145a and second hole portions 147a and 147b).

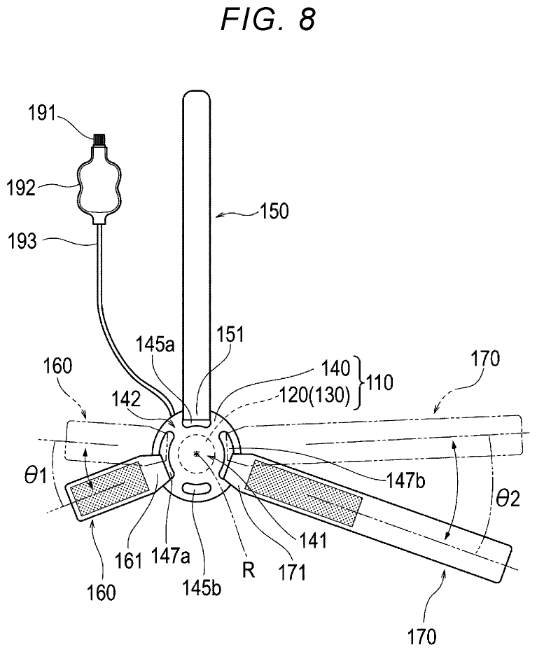

In the hemostatic device 100, as will be described later, of the three band bodies of the first band body 150, the second band body 160, and the third band body 170, the two band bodies of the second band body 160 and the third band body 170 are configured to be capable of being disposed so as to radially change in angle about a pressing portion 120 positioned in a first region 141. Accordingly, of the plurality of hole portions provided in the second region 142, the two hole portions forming the pair of second hole portions 147a and 147b are larger than the widths of the second band body 160 and the third band body 170 and configured in a non-linear shape. It is preferable that the shape of the hole portions 147a and 147b is a curved shape. With the second band body 160 and the third band body 170 disposed so as to be respectively rewound through the hole portions 147a and 147b, some of the constituent members from which the second band body 160 and the third band body 170 may be fabricated can be connected by a method such as fusion. As a result, the state of connection to each of the hole portions 147a and 147b can be maintained. Since the size of the hole portions 147a and 147b (extension-direction length of the hole portions 147a and 147b) is larger than the width of the band bodies 160 and 170, the hole portions 147a and 147b are provided with a play (i.e., a space) that enables the band bodies 160 and 170 to slide in the right-left direction. In addition, since the hole portions 147a and 147b have a non-linear shape such as a curved shape and a bent shape, the extension-direction angles of the band bodies 160 and 170 with respect to the pressing portion 120 (angles in the radial direction about the pressing portion 120) can be changed by changing the positions of the band bodies 160 and 170 in the hole portions 147a and 147b. As illustrated in FIG. 8, in the hemostatic device 100, of the three band bodies of the first band body 150, the second band body 160, and the third band body 170, the two band bodies of the second band body 160 and the third band body 170 are configured to be slidable along the hole portions 147a and 147b about the pressing portion 120. In addition, the two band bodies of the second band body 160 and the third band body 170 can be disposed so as to radially change in angle about a center point R provided in the first region 141.

As illustrated in FIG. 14, the first band body 150 can be disposed so as to be hooked on an interdigital portion fb positioned between two adjacent fingers (for example, the thumb and the index finger) when the hemostatic device 100 is mounted onto the patient's right hand H1.

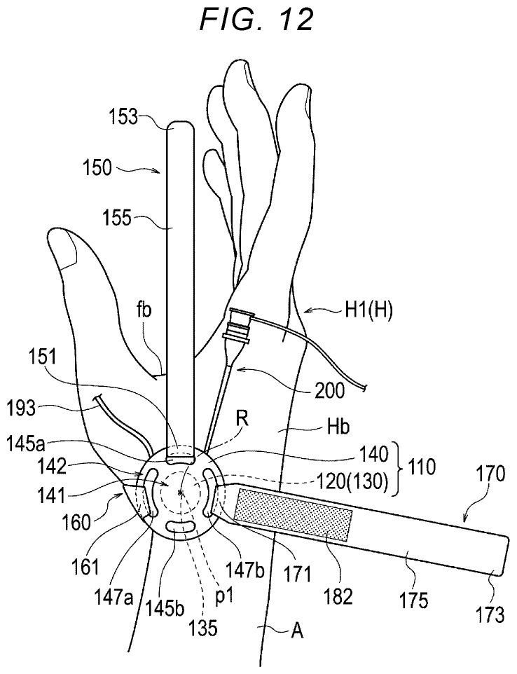

As illustrated in FIGS. 12, 13, and 14, the second band body 160 and the third band body 170 can be disposed so as to be wound along the outer circumference of the right hand H1 when the hemostatic device 100 is mounted onto the patient's right hand H1.

Pressing Member

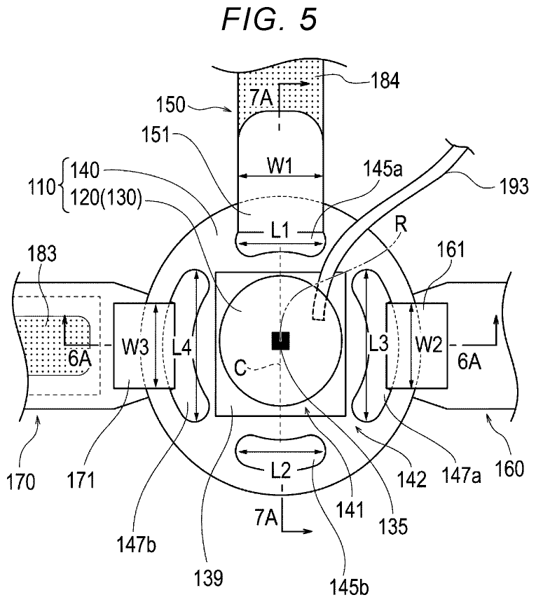

As illustrated in FIGS. 5, 6, and 7, the pressing member 110 has the pressing portion 120 configured to compress the first puncture site p1 formed in the patient's right hand H1 and the support member 140 configured to fix the pressing portion 120.

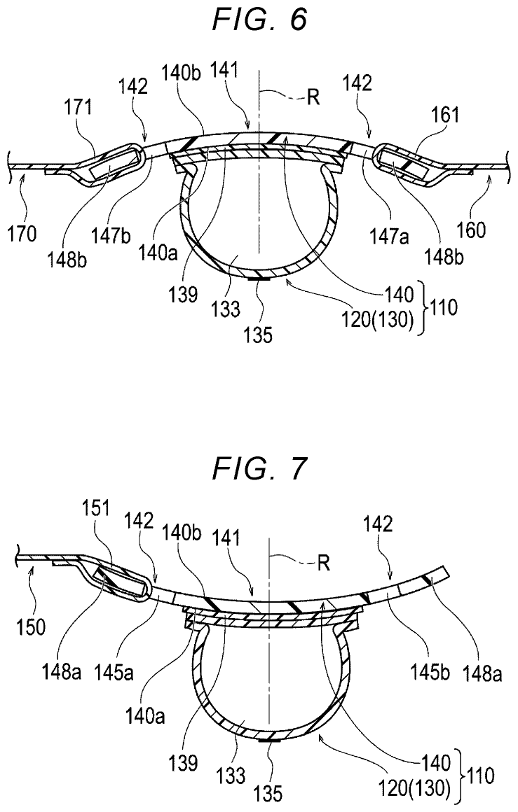

As illustrated in FIGS. 6 and 7, the pressing portion 120 can be configured by, for example, an inflatable member 130 provided with a lumen 133 into which a fluid such as air is capable of flowing. Note that FIGS. 6 and 7 illustrate cross-sectional views of a state where the inflatable member 130 is inflated. In addition, FIGS. 6 and 7 illustrate partial cross-sectional views obtained by turning upside down the cross sections along the arrows 6A-6A and 7A-7A illustrated in FIG. 5.

The inflatable member 130 can be configured by, for example, a resin balloon. A tube 193, which will be described later, is connected to the lumen 133 of the inflatable member 130.

As illustrated in FIGS. 6, 7, 15, and 16, the inflatable member 130 is disposed on one surface 140a side of the support member 140. The one surface 140a of the support member 140 is disposed on the body surface side of the patient's hand H when the hemostatic device 100 is mounted on the patient's hand H. In addition, the other surface 140b of the support member 140 is positioned on the side opposite to the one surface 140a.

As illustrated in FIGS. 6 and 7, the inflatable member 130 can be connected to the support member 140 via a predetermined connection member 139.

The connection member 139 can be configured by, for example, a resin plate. The connection member 139 is connected to the one surface 140a of the support member 140. The inflatable member 130 is connected to the connection member 139.

Fusion, adhesion, or the like can be adopted for the connection between the connection member 139 and the support member 140 and the connection between the inflatable member 130 and the connection member 139. Note that the inflatable member 130 may be connected to the one surface 140a of the support member 140 directly and not via the connection member 139.

The inflatable member 130 is circular in the plan views illustrated in FIGS. 1 to 5. However, the shape of the inflatable member 130 in a plan view is not limited to a circle. In addition, the pre-inflation and post-inflation cross-sectional shapes of the inflatable member 130, constituent materials from which the inflatable member 130 and the specific structure of the inflatable member 130 may be fabricated are not particularly limited.

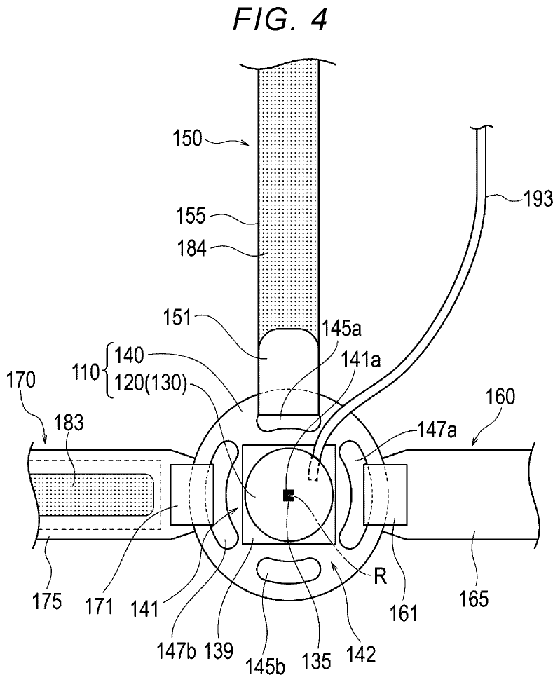

As illustrated in FIGS. 4, 5, 6, 7, 15, and 16, a marker 135 for aligning the inflatable member 130 with the first puncture site p1 is disposed on the inflatable member 130.

The marker 135 is disposed on the surface of the inflatable member 130 on the side opposite to the surface on the side where the support member 140 is disposed (surface disposed on the body surface side of the patient's hand H when the hemostatic device 100 is mounted on the patient's hand H).

As illustrated in FIG. 5, the marker 135 is disposed at the substantially central position in the plane direction of the inflatable member 130. In addition, the marker 135 is disposed so as to overlap the substantially central position in the plane direction of the support member 140.

The marker 135 can be formed by, for example, a rectangular marker with the entire marker 135 colored. Note that the specific shape and color of the marker 135, how to form the marker 135, the position of the marker 135, and so on are not particularly limited. For example, the marker 135 may be configured from a transparent center portion and a colored and linear frame portion surrounding the center portion. In addition, for example, the marker 135 may be provided on the support member 140.

The specific configuration of the pressing portion 120 is not limited insofar as a compressive force can be applied to the first puncture site p1 formed in the patient's right hand H1. The pressing portion 120 can be configured by, for example, a member configured by, for example, a resin material such as plastic or a gel, a member containing a gel declining in moisture content with time to gradually decrease the compressive force, an elastic material such as a spongy substance, an aggregate of fibers such as cotton, a metal, a member having a predetermined three-dimensional shape (for example, spherical, ellipsoid, and triangular pyramid), or an appropriate combination thereof.

Support Member

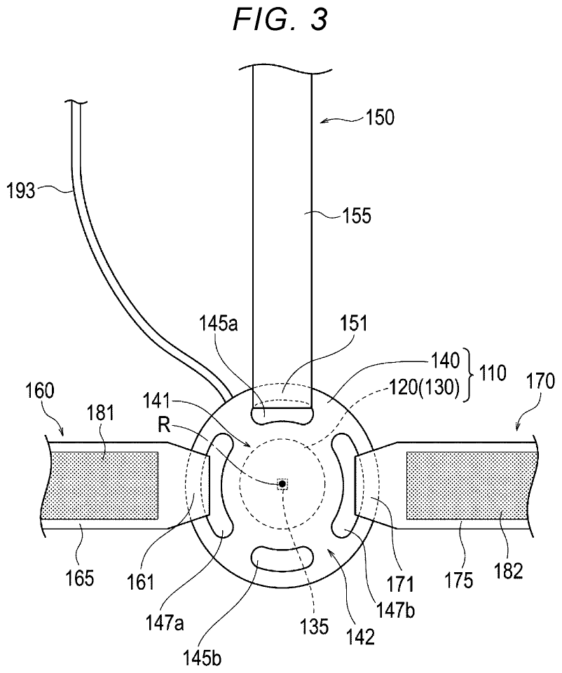

As illustrated in FIGS. 3, 5, 6, and 7, the support member 140 has the first region 141 where the inflatable member 130 is disposed and the second region 142 positioned outside the first region 141 and configured such that the first band body 150, the second band body 160, and the third band body 170 can be connected.

The support member 140 is circular in the plan view illustrated in FIG. 5.

The pressing portion 120 overlaps the first region 141 in the plan view illustrated in FIG. 5. The second region 142 is positioned outside the first region 141 in the plan view illustrated in FIG. 5.

Note that any first region 141 can be defined based on the outer shape or size of the pressing portion 120 disposed on the support member 140. In addition, the second region 142 can be defined based on the positional relationship relative to the first region 141. Accordingly, the first region 141 and the second region 142 can be appropriately changed in accordance with the outer shape or size of the pressing portion 120 disposed on the support member 140.

As illustrated in FIGS. 5, 6, 7, and 8, the center point R, which is the center when the second band body 160 and the third band body 170 slide along the second hole portions 147a and 147b and are disposed radially, is positioned in the first region 141.

As illustrated in FIGS. 5, 6, 7, 9, and 10, a pair of the first hole portion 145a and a first hole portion 145b facing each other with the pressing portion 120 interposed therebetween and the pair of second hole portions 147a and 147b facing each other with the pressing portion 120 interposed therebetween at positions different from the pair of first hole portions 145a and 145b are formed in the second region 142.

In the description of the present specification, the first hole portion 145a is also referred to as “one first hole portion 145a”. In addition, the first hole portion 145b is also referred to as “the other first hole portion 145b”. In addition, the second hole portion 147a is also referred to as “one second hole portion 147a”. In addition, the second hole portion 147b is also referred to as “the other second hole portion 147b”.

As illustrated in FIG. 5, the one first hole portion 145a and the other first hole portion 145b are disposed on the distal side (fingertip side) of the hand H or the proximal side (forearm A side) of the hand H with the pressing portion 120 interposed between the one first hole portion 145a and the other first hole portion 145b. In the present embodiment, when the hemostatic device 100 is mounted onto the patient's right hand H1, the one first hole portion 145a is disposed closer to the distal side of the hand H than the pressing portion 120 and the other first hole portion 145b is disposed closer to the proximal side of the hand H than the pressing portion 120 (see FIG. 14).

Note that the one first hole portion 145a and the other first hole portion 145b can be disposed with the positional relationship of the one first hole portion 145a and the other first hole portion 145b with respect to the pressing portion 120 interchanged in a case where the position of attachment of the hemostatic device 100 to the patient's hand H is changed as described in the modification example to be described later (see FIG. 24). In other words, the one first hole portion 145a may be disposed closer to the proximal side of the hand H than the pressing portion 120 and the other first hole portion 145b may be disposed closer to the distal side of the hand H than the pressing portion 120.

As illustrated in FIGS. 3, 4, and 5, the one first hole portion 145a and the other first hole portion 145b are disposed on a virtual circle along the outer shape of the support member 140. In the present embodiment, the support member 140 is circular in the plan view illustrated in FIG. 5. Accordingly, the one first hole portion 145a and the other first hole portion 145b are disposed along the circumferential direction of the second region 142 positioned outside the first region 141 in the support member 140.

The above “disposed on a virtual circle” means that the one first hole portion 145a and the other first hole portion 145b are disposed with a gap between the one first hole portion 145a and the other first hole portion 145b and at any positions occupying a part of a hole with a case assumed where the hole is formed continuous in the circumferential direction of the second region 142 of the support member 140. The same definition as above applies to the positional relationship between the one second hole portion 147a and the other second hole portion 147b, which are disposed on a virtual circle and will be described later.

As illustrated in FIGS. 3, 4, 5, and 7, the first band body 150 can be connected to the one first hole portion 145a.

In the present embodiment, a width W1 of a first one end portion 151 of the first band body 150 (width in a direction orthogonal to the extension direction of the first band body 150) is substantially equal to a hole length L1 of the one first hole portion 145a (see FIG. 5). Accordingly, the sliding of the first band body 150 about the center point R provided in the first region 141 of the support member 140 is limited in a state where the first band body 150 is connected to the support member 140 via the one first hole portion 145a. In other words, the sliding of the first band body 150 about the pressing portion 120 is limited in a state where the first band body 150 is connected to the support member 140 via the one first hole portion 145a.

Note that in the present specification, the hole lengths of the hole portions 145a, 145b, 147a, and 147b mean the straight-line distances between the end portions of the hole portions 145a, 145b, 147a, and 147b positioned in the circumferential direction of the support member 140, respectively.

The width W1 of the one first hole portion 145a is formed to be substantially equal to a hole length L2 of the other first hole portion 145b (see FIG. 5). Accordingly, in a case where the first band body 150 is connected to the support member 140 via the other first hole portion 145b, the sliding of the first band body 150 about the center point R provided in the first region 141 of the support member 140 is limited (see FIG. 24 of the modification example to be described later). In other words, in a case where the first band body 150 is connected to the support member 140 via the other first hole portion 145b, the sliding of the first band body 150 about the pressing portion 120 is limited.

As illustrated in FIGS. 3, 4, and 5, the one second hole portion 147a and the other second hole portion 147b are disposed with the pressing portion 120 interposed between the one second hole portion 147a and the other second hole portion 147b in a direction intersecting a straight line connecting the one first hole portion 145a and the other first hole portion 145b (virtual line C illustrated in FIG. 5).

As illustrated in FIGS. 3 and 4, the one second hole portion 147a and the other second hole portion 147b are disposed on a virtual circle along the outer shape of the support member 140.

As for the hemostatic device 100, in a case where the one first hole portion 145a is disposed closer to the distal side of the patient's right hand H1 than the pressing portion 120 and the other first hole portion 145b is disposed closer to the proximal side of the patient's right hand H1 than the pressing portion 120 as illustrated in FIG. 12, the one second hole portion 147a can be disposed on the inside in the circumferential direction of the patient's right hand H1 (side where the patient's torso is disposed) and the other second hole portion 147b can be disposed on the outside in the circumferential direction of the patient's right hand H1.

A hole length L3 of the one second hole portion 147a is longer than each of the hole length L1 of the one first hole portion 145a and the hole length L2 of the other first hole portion 145b (see FIG. 5). Likewise, a hole length L4 of the other second hole portion 147b is longer than each of the hole length L1 of the one first hole portion 145a and the hole length L2 of the other first hole portion 145b. The hole length L3 of the one second hole portion 147a and the hole length L4 of the other second hole portion 147b are formed to be substantially equal to each other.

As illustrated in FIGS. 3, 4, 5, and 6, the second band body 160 is connected to the one second hole portion 147a.

A width W2 of a second one end portion 161 of the second band body 160 (width in a direction orthogonal to the extension direction of the second band body 160) is shorter than the hole length L3 of the one second hole portion 147a (see FIG. 5). Accordingly, in a state where the second band body 160 is connected to the pressing member 110 via the one second hole portion 147a as illustrated in FIG. 8, the second band body 160 is capable of sliding about the center point R provided in the first region 141 of the support member 140 so as to radially change in angle in the range of a predetermined slide angle 81.

A slide angle 81 of the second band body 160 can be defined by the width W2 of the second one end portion 161 of the second band body 160 and the hole length L3 of the one second hole portion 147a. Although the slide angle 81 of the second band body 160 is not particularly limited, the slide angle 81 of the second band body 160 can be set to, for example, 1° to 45° about the center point R provided in the first region 141.

As illustrated in FIGS. 3, 4, 5, and 6, the third band body 170 is connected to the other second hole portion 147b.

A width W3 of a third one end portion 171 of the third band body 170 (width in a direction orthogonal to the extension direction of the third band body 170) is shorter than the hole length L4 of the other second hole portion 147b (see FIG. 5). Accordingly, in a state where the third band body 170 is connected to the other second hole portion 147b as illustrated in FIG. 8, the third band body 170 is capable of sliding about the center point R provided in the first region 141 of the support member 140 so as to radially change in angle in the range of a predetermined slide angle 82.

A slide angle 82 of the third band body 170 can be defined by the width W3 of the third one end portion 171 of the third band body 170 and the hole length L4 of the other second hole portion 147b. Although the slide angle 82 of the third band body 170 is not particularly limited, the slide angle 82 of the third band body 170 can be set to, for example, 1° to 45° about the center point R provided in the first region 141.

Note that as illustrated in FIG. 5, each of the first hole portions 145a and 145b and each of the second hole portions 147a and 147b can be disposed on a virtual concentric circle about the center point R provided in the first region 141 of the support member 140. However, each of the first hole portions 145a and 145b and each of the second hole portions 147a and 147b may not be disposed on the virtual concentric circle.

In addition, the one first hole portion 145a and the other first hole portion 145b may be different in shape or hole length. In addition, the one second hole portion 147a and the other second hole portion 147b may be different in shape or hole length.

In addition, the hole length L1 of the one first hole portion 145a and/or the hole length L2 of the other first hole portion 145b may be formed to be larger than the width W1 of the first one end portion 151 of the first band body 150. In the case of this configuration, in a state where the first band body 150 is connected to the support member 140 via the one first hole portion 145a or the other first hole portion 145b, the first band body 150 is capable of sliding about the center point R provided in the first region 141 of the support member 140 so as to radially change in angle.

As illustrated in FIGS. 7, 9, 10, and 16, a first curved region 148a convexly curved toward a side away from the pressing portion 120 disposed on the one surface 140a of the support member 140 is formed at the part of the second region 142 of the support member 140 where the pair of first hole portions 145a and 145b are disposed.

The above “side away from the pressing portion 120” is a side away from the body surface of the patient's hand H when the hemostatic device 100 is mounted on the patient's hand H as illustrated in FIG. 16 (upper side in FIG. 16).

As illustrated in FIGS. 6, 9, 10, and 15, a second curved region 148b convexly curved toward the side of the pressing portion 120 disposed on the one surface 140a of the support member 140 is formed at the part of the second region 142 of the support member 140 where the pair of second hole portions 147a and 147b are disposed.

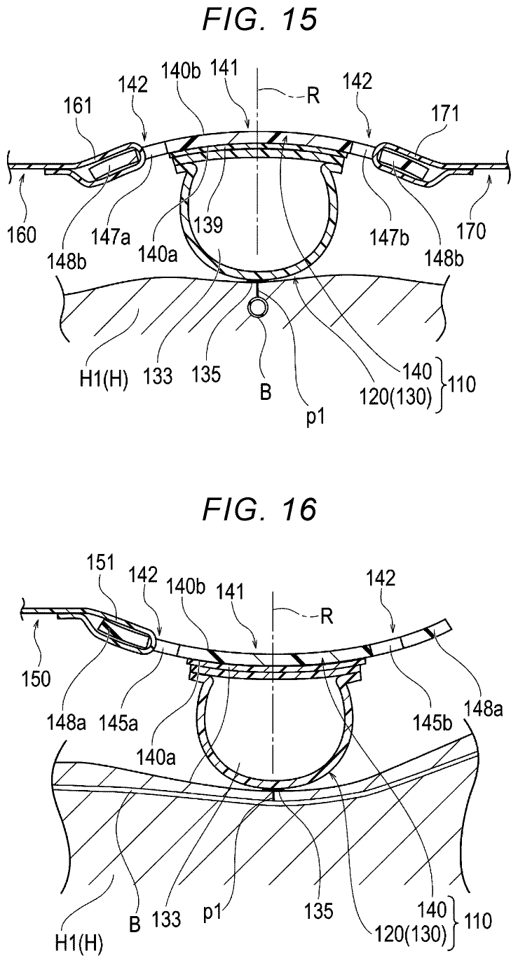

The above “pressing portion 120 side” is a side close to the body surface of the patient's hand H when the hemostatic device 100 is mounted on the patient's hand H as illustrated in FIG. 15 (lower side in FIG. 15).

It is preferable that the support member 140 is configured by a material with a predetermined hardness. In a case where the support member 140 is configured to have a predetermined hardness, the support member 140 is capable of pressing the pressing portion 120 against the patient's right hand H1 when the pressing portion 120 applies a compressive force to the first puncture site p1 formed in the patient's right hand H1 as illustrated in FIGS. 15 and 16. As a result, it is possible to prevent the pressing portion 120 from floating up from the patient's right hand H1.

Usable as constituent materials for the support member 140 having the hardness described above are, for example, acrylic resin, polyvinyl chloride (hard polyvinyl chloride in particular), polyolefin such as polyethylene, polypropylene, and polybutadiene, polystyrene, poly-(4-methylpentene-1), polycarbonate, ABS resin, polymethylmethacrylate (PMMA), polyacetal, polyacrylate, polyacrylonitrile, polyvinylidene fluoride, ionomer, acrylonitrile-butadiene-styrene copolymer, and polyethylene terephthalate (PET).

The respective parts of the pressing portion 120 and the support member 140 mutually overlapping in the plan views illustrated in FIGS. 4 and 5 can be transparently formed. In a case where the pressing portion 120 and the support member 140 are configured in this manner, as illustrated in FIGS. 12, 13, and 14, when the hemostatic device 100 is mounted onto the patient's right hand H1, an operator can visually confirm the position of the marker 135 and/or the first puncture site p1 with greater ease via the pressing portion 120 and the support member 140. Note that the above “transparent” includes colored transparent, colorless transparent, and translucent.

Band Body

As illustrated in FIGS. 1, 2, 3, and 4, the first band body 150 has a first main body portion 155, the first one end portion 151 configured to be connectable to the one first hole portion 145a of the support member 140, and a free first other end portion 153.

Note that the “free other end portion” in the present specification means an end portion having no direct or indirect connection relationship with another member in a state where the hemostatic device 100 is not mounted (state where the hemostatic device is not mounted on the patient).

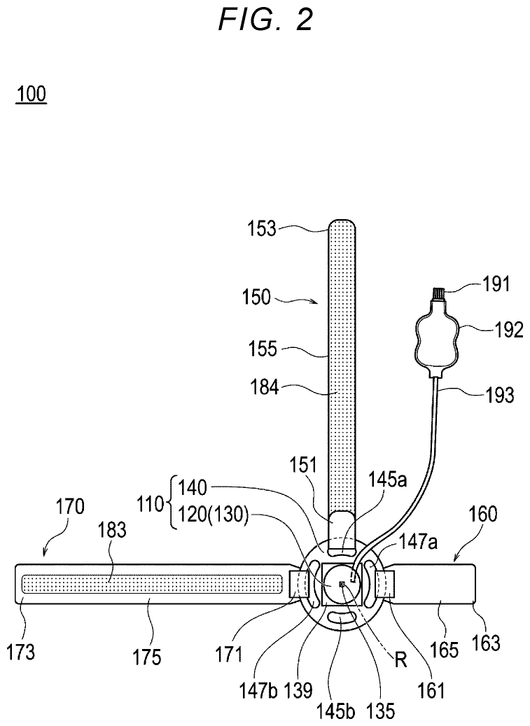

As illustrated in FIGS. 1 and 2, the first main body portion 155 extends along the longitudinal direction of the first band body 150.

As illustrated in FIG. 7, the first one end portion 151 of the first band body 150 can be disposed so as to be inserted through and wound around the one first hole portion 145a of the support member 140. The first band body 150 is connected to the support member 140 via the first one end portion 151 and the one first hole portion 145a.

As described above, in the present embodiment, the width W1 of the first one end portion 151 of the first band body 150 is formed to be substantially equal to the hole length L1 of the one first hole portion 145a (see FIG. 5). Accordingly, in a state where the first band body 150 is connected to the support member 140, the sliding of the first band body 150 about the center point R provided in the first region 141 of the support member 140 is limited.

Note that the structure of connection between the first one end portion 151 of the first band body 150 and the support member 140 is not particularly limited. For example, a fixing member (for example, a hook-and-loop fastener) capable of maintaining and releasing the state of winding around the one first hole portion 145a can be disposed on the first one end portion 151 of the first band body 150. In a case where the first band body 150 is configured in this manner, the first band body 150 can be connected to and separated from the support member 140.

As illustrated in FIGS. 1, 2, 3, and 4, the second band body 160 has a second main body portion 165, the second one end portion 161 configured to be connectable to the one second hole portion 147a of the support member 140, and a free second other end portion 163.

As illustrated in FIGS. 1 and 2, the second main body portion 165 extends along the longitudinal direction of the second band body 160.

As illustrated in FIG. 6, the second one end portion 161 of the second band body 160 can be disposed so as to be inserted through and wound around the one second hole portion 147a of the support member 140. The second band body 160 is connected to the support member 140 via the second one end portion 161 and the one second hole portion 147a.

As described above, the width W2 of the second one end portion 161 of the second band body 160 is shorter than the hole length L3 of the one second hole portion 147a (see FIG. 5). Accordingly, in a state where the second band body 160 is connected to the support member 140, the second band body 160 is capable of sliding about the center point R provided in the first region 141 of the support member 140 so as to radially change in angle (see FIG. 8).

Note that the structure of connection between the second one end portion 161 of the second band body 160 and the support member 140 is not particularly limited. For example, a fixing member (for example, a hook-and-loop fastener) capable of maintaining and releasing the state of winding around the one second hole portion 147a can be disposed on the second one end portion 161 of the second band body 160. In a case where the second band body 160 is configured in this manner, the second band body 160 can be connected to and separated from the support member 140.

As illustrated in FIGS. 1, 2, 3, and 4, the third band body 170 has a third main body portion 175, the third one end portion 171 configured to be connectable to the other second hole portion 147b of the support member 140, and a free third other end portion 173.

As illustrated in FIGS. 1 and 2, the third main body portion 175 extends along the longitudinal direction of the third band body 170.

As illustrated in FIG. 6, the third one end portion 171 of the third band body 170 can be disposed so as to be inserted through and wound around the other second hole portion 147b of the support member 140. The third band body 170 is connected to the support member 140 via the third one end portion 171 and the other second hole portion 147b.

As described above, the width W3 of the third one end portion 171 of the third band body 170 is shorter than the hole length L4 of the other second hole portion 147b (see FIG. 5). Accordingly, in a state where the third band body 170 is connected to the support member 140, the third band body 170 is capable of sliding about the center point R provided in the first region 141 of the support member 140 so as to radially change in angle (see FIG. 8).

Note that the structure of connection between the third one end portion 171 of the third band body 170 and the support member 140 is not particularly limited. For example, a fixing member (for example, a hook-and-loop fastener) capable of maintaining and releasing the state of winding around the other second hole portion 147b can be disposed on the third one end portion 171 of the third band body 170. In a case where the third band body 170 is configured in this manner, the third band body 170 can be connected to and separated from the support member 140.

Although constituent materials from which each of the band bodies 150, 160, and 170 may be fabricated are not particularly limited, each of the band bodies 150, 160, and 170 can be configured by, for example, vinyl chloride resin, polyurethane resin, polyester resin, or the like. In addition, each of the band bodies 150, 160, and 170 is not particularly limited in shape, length, thickness, and so on.

Fixing Member

As illustrated in FIGS. 1, 2, 3, and 4, the hemostatic device 100 includes the four fixing members of a first fixing member 181, a second fixing member 182, a third fixing member 183, and a fourth fixing member 184.

As illustrated in FIGS. 1 and 3, the first fixing member 181 is disposed on the outer surface of the second band body 160. The second fixing member 182 is disposed on the outer surface of the third band body 170.

In addition, as illustrated in FIGS. 2 and 4, the third fixing member 183 is disposed on the inner surface of the third band body 170. The fourth fixing member 184 is disposed on the inner surface of the first band body 150.

Note that the inner surface of each of the band bodies 150, 160, and 170 is a surface disposed on the body surface side of the patient when the hemostatic device 100 is mounted on the patient, and the outer surface of each of the band bodies 150, 160, and 170 is a surface positioned on the side opposite to the inner surface.

The first fixing member 181 and the second fixing member 182 are configured on the male side of a hook-and-loop fastener. The third fixing member 183 and the fourth fixing member 184 are configured on the female side of the hook-and-loop fastener. The hook-and-loop fastener in the present specification is a fastener detachable in terms of surface and is, for example, Magic Tape® or Velcro®.

Note that the specific structure of each of the fixing members 181, 182, 183, and 184 is not limited insofar as the pressing portion 120 can be fixed to the right hand H1 by interconnecting the respective band bodies 150, 160, and 170 with the hemostatic device 100 disposed on the patient's right hand H1. For example, a partial omission of fixing member installation, a change in fixing member disposition position in each of the band bodies 150, 160, and 170, and so on can be performed in any manner. In addition, in a case where each of the fixing members 181, 182, 183, and 184 is configured by a hook-and-loop fastener, the male side and the female side of the hook-and-loop fastener may be interchanged. In addition, each of the fixing members 181, 182, 183, and 184 may be, for example, a snap, a button, a clip, a hole portion-formed frame member, or the like. In addition, for example, a fixing member (third fixing member) may be disposed on the inner surface of the second band body 160 as well.

Injection Unit

As illustrated in FIGS. 1 and 2, the hemostatic device 100 has an injection unit 191 for fluid injection into the inflatable member 130.

The injection unit 191 is configured by a connector incorporating a check valve. A syringe can be connected to the injection unit 191.

A cushioning member 192 having an inflatable space is disposed between the injection unit 191 and the inflatable member 130. The cushioning member 192 is configured by a flexible bag-shaped member in which a space is formed. Note that the cushioning member 192 may be provided with an arrow-shaped marker indicating the direction of syringe insertion into the injection unit 191.

The injection unit 191 is connected to one end side of the cushioning member 192. The lumen of the injection unit 191 communicates with the space of the cushioning member 192. However, while the check valve incorporated in the injection unit 191 is closed, the communication between the lumen of the injection unit 191 and the space of the cushioning member 192 is interrupted.

The flexible tube 193 is connected to the other end side of the cushioning member 192. The lumen of the tube 193 communicates with the space of the cushioning member 192. In addition, the other end portion of the tube 193 on the side opposite to the one end portion connected to the cushioning member 192 is connected to the inflatable member 130. The lumen of the tube 193 communicates with the lumen 133 of the inflatable member 130.

In inflating the inflatable member 130, an operator inserts the tip tube portion of a syringe into the injection unit 191 and opens the check valve. The operator injects the air in the syringe into the lumen 133 of the inflatable member 130 by pushing the pusher of the syringe with the check valve of the injection unit 191 open.

The inflatable member 130 inflates when the air is injected into the lumen 133 of the inflatable member 130. When the inflatable member 130 inflates, the cushioning member 192 communicating with the lumen 133 of the inflatable member 130 via the tube 193 inflates. By visually confirming the inflation of the cushioning member 192, the operator can rather easily grasp that the inflatable member 130 has inflated without air leakage.

In contracting the inflatable member 130, the operator inserts the tip tube portion of the syringe into the injection unit 191 and pulls the pusher of the syringe. By performing the above operation, the operator can discharge the air in the lumen 133 of the inflatable member 130 to the syringe.

Examples of Use of Hemostatic Device

Next, a first example of use of the hemostatic device 100 will be described with reference to FIGS. 12 to 16.

In the first example of use, the procedure of use of the hemostatic device 100 at the time of hemostasis at the first puncture site p1 formed in the patient's right hand H1 will be described.

FIG. 12 illustrates a state where various procedures are completed with the sheath tube of the introducer 200 inserted in the first puncture site p1.

As illustrated in FIG. 12, an operator disposes the pressing member 110 so as to overlap the back Hb of the patient's right hand H1. At this time, the operator can appropriately position the pressing member 110 at the first puncture site p1 by disposing the marker 135 at the first puncture site p1 while visually confirming the position of the marker 135 formed on the inflatable member 130.

Note that after completing a procedure using the introducer 200 and before mounting the hemostatic device 100 onto the patient's right hand H1, the operator may pull out a part of the sheath tube of the introducer 200 from the first puncture site p1 formed in the patient's right hand H1. For example, with the sheath tube of the introducer 200 placed in the blood vessel B, the operator can start the work of mounting the hemostatic device 100 after pulling out the sheath tube by, for example, approximately 2 cm to 3 cm to the hand side of the operator.

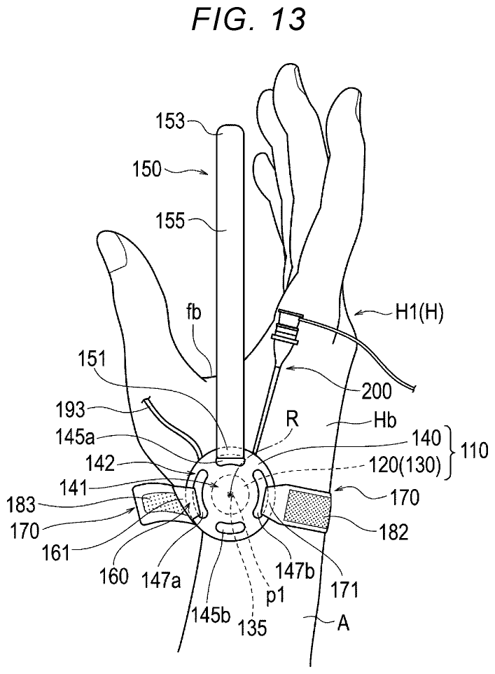

As illustrated in FIGS. 12 and 13, the operator winds the second band body 160 and the third band body 170 along the outer circumference of the patient's right hand H1. The operator can respectively fix the second band body 160 and the third band body 170 via the fixing members 181 and 183 by bringing the third fixing member 183 (see FIG. 2) disposed on the inner surface of the third band body 170 into contact with the first fixing member 181 (see FIG. 1) disposed on the outer surface of the second band body 160.

In winding the second band body 160 and the third band body 170 along the outer circumference of the patient's right hand H1, the operator can radially slide the second band body 160 or radially slide the third band body 170 about the center point R provided in the first region 141 of the support member 140. By sliding the second band body 160 and the third band body 170 so as to radially change in angle, the operator can adjust the respective positions where the band bodies 160 and 170 are wound around the patient's right hand H1. For example, the operator can respectively slide the band bodies 160 and 170 in the ranges of the slide angles 81 and 82 (see FIG. 8) such that each of the band bodies 160 and 170 is wound around the patient's right hand H1 at a position closer to the forearm A side (proximal side) than the first puncture site p1.

As illustrated in FIG. 14, the operator disposes a part of the first band body 150 on the palm side of the patient's right hand H1 while passing the first band body 150 through the interdigital portion fb positioned between the thumb and the index finger of the patient's right hand H1. At this time, the operator can respectively fix the first band body 150 and the third band body 170 via the fixing members 182 and 184 by bringing the fourth fixing member 184 (see FIG. 2) disposed on the inner surface of the first band body 150 into contact with the second fixing member 182 (see FIG. 1) disposed on the outer surface of the third band body 170.

The operator can effectively prevent a positional deviation of the hemostatic device 100 from the patient's right hand H1 by disposing the second band body 160 and the third band body 170 so as to be wound along the outer circumference of the patient's right hand H1 and, further, disposing a part of the first band body 150 to be hooked on the interdigital portion fb between the thumb and the index finger of the patient's right hand H1 as described above.

The operator inflates the inflatable member 130 by injecting air into the inflatable member 130 with a syringe connected to the injection unit 191. As illustrated in FIGS. 15 and 16, in the hemostatic device 100, the inflatable member 130 applies a compressive force to the first puncture site p1 in the patient's right hand H1 when the inflatable member 130 inflates.

As illustrated in FIGS. 14 and 16, in mounting the hemostatic device 100 onto the patient's hand H, the operator can dispose the first curved region 148a formed near the one first hole portion 145a at a distal side position of the patient's right hand H1. In addition, the operator can dispose the first curved region 148a formed near the other first hole portion 145b at a proximal side position of the patient's right hand H1. By disposing the hemostatic device 100 in this manner, when the patient twists his or her wrist to move the right hand H1 in the up-down or right-left direction with the hemostatic device 100 mounted on the patient's right hand H1, the first curved region 148a helps prevent the peripheral edge portion of the support member 140 from abutting against the patient's right hand H1. Likewise, in the hemostatic device 100, when the patient moves the right hand H1 as described above, the first curved region 148a formed near the other first hole portion 145b helps prevent the peripheral edge portion of the support member 140 from abutting against the patient's right hand H1. As a result, while hemostasis is performed with the hemostatic device 100 at the first puncture site p1 formed in the patient's right hand H1, it is possible to prevent the patient from feeling discomfort or pain due to the peripheral edge portion of the support member 140 abutting against or biting into the patient's right hand H1.

In addition, as illustrated in FIGS. 14 and 15, the operator in mounting the hemostatic device 100 onto the patient's hand H can fix the pressing portion 120 to the patient's right hand H1 such that the second curved region 148b formed on the support member 140 is disposed along a part of the outer circumference of the patient's right hand H1. When the pressing portion 120 inflates with the hemostatic device 100 mounted on the patient's right hand H1 in this manner, the second curved region 148b of the support member 140 presses the pressing portion 120 along a part of the outer circumference of the patient's right hand H1. As a result, the hemostatic device 100 is capable of helping prevent the pressing portion 120 from floating up from the patient's right hand H1. Accordingly, the hemostatic device 100 is capable of effectively applying a compressive force to the first puncture site p1 by the inflatable member 130.

As illustrated in FIG. 14, after inflating the inflatable member 130, the operator removes the sheath tube of the introducer 200 from the first puncture site p1 formed in the patient's right hand H1. The operator confirms that there is no bleeding from the first puncture site p1 formed in the patient's right hand H1 while hemostasis is performed using the hemostatic device 100. The operator adjusts the amount of air injection into the inflatable member 130 in a case where there is bleeding from the first puncture site p1 formed in the patient's right hand H1.

By the above procedure, the operator can perform hemostasis at the first puncture site p1 formed in the patient's right hand H1 using the hemostatic device 100.

FIG. 17 illustrates a second example of use of the hemostatic device 100. The second example of use is an example of use of the hemostatic device 100 at the time of hemostasis at the second puncture site p2 formed in the patient's right hand H1.

As illustrated in FIG. 17, the operator mounts the hemostatic device 100 onto the patient's right hand H1 in performing hemostasis at the second puncture site p2 formed in the patient's right hand H1. The second puncture site p2 formed in the patient's right hand H1 is positioned closer to the distal side of the patient's right hand H1 than the first puncture site p1 described above (see FIG. 11). In winding each of the band bodies 160 and 170 around the patient's right hand H1, the operator slides each of the band bodies 160 and 170 about the center point R provided in the first region 141 of the support member 140. For example, the operator slides each of the band bodies 160 and 170 such that each of the band bodies 160 and 170 is wound around the patient's right hand H1 at a position closer to the forearm A side (proximal side) than the first puncture site p1. The operator can help prevent the distal side part of the patient's right hand H1 from being restrained by the respective band bodies 160 and 170 by winding the respective band bodies 160 and 170 at the proximal side position of the patient's right hand H1.

FIG. 19 illustrates a third example of use of the hemostatic device 100. In addition, FIG. 20 illustrates a fourth example of use of the hemostatic device 100. The third example of use is an example of use of the hemostatic device 100 at the time of hemostasis at the third puncture site p3 formed in the patient's left hand H2. The fourth example of use is an example of use of the hemostatic device 100 at the time of hemostasis at the fourth puncture site p4 formed in the patient's left hand H2.

Also in the third example of use and the fourth example of use, the hemostatic device 100 can be mounted onto the patient's left hand H2 by the same procedure as in the first example of use and the second example of use described above. An operator can adjust the position of winding each of the band bodies 160 and 170 around the left hand H2 by sliding each of the band bodies 160 and 170 about the center point R provided in the first region 141 of the support member 140 in accordance with the position of each of the puncture sites p3 and p4 in the patient's left hand H2. By adjusting the respective winding positions of the band bodies 160 and 170 around the left hand H2, the operator can help prevent the distal side part of the patient's left hand H2 from being restrained by the respective band bodies 160 and 170 when hemostasis is performed at each of the puncture sites p3 and p4.

As described through each example of use, the hemostatic device 100 can be mounted onto both the patient's right hand H1 and the patient's left hand H2. In addition, the hemostatic device 100 can be mounted onto the patient's hand H such that the distal side part of the patient's hand H is not restrained by the respective band bodies 160 and 170 in both a case where the hemostatic device 100 is used for hemostasis at each of the puncture sites p1 and p2 formed at different positions in the patient's right hand H1 and a case where the hemostatic device 100 is used for hemostasis at each of the puncture sites p3 and p4 formed at different positions in the patient's left hand H2.

The hemostatic device 100 according to the present embodiment described above includes: the pressing member 110 configured to compress the first puncture site p1 formed in a patient; the first band body 150 configured to be connectable to the pressing member 110; the second band body 160 configured to be connectable to the pressing member 110; and the third band body 170 configured to be connectable to the pressing member 110. The pressing member 110 has the pressing portion 120 configured to compress the first puncture site p1 and the support member 140 configured to fix the pressing portion 120. The support member 140 has the first region 141 where the pressing portion 120 is disposed and the second region 142 positioned outside the first region 141 and configured such that the first band body 150, the second band body 160, and the third band body 170 are connectable. The first band body 150, the second band body 160, and the third band body 170 are configured to be respectively connectable to the plurality of hole portions provided in the second region 142 (first hole portion 145a and second hole portions 147a and 147b). Of the plurality of hole portions, the second hole portions 147a and 147b are larger than the widths of the band bodies 160 and 170 (the width of the second one end portion 161 of the second band body 160 is longer than the hole length of the one second hole portion 147a and the width of the third one end portion 171 of the third band body 170 is longer than the hole length of the other second hole portion 147b) and configured in a non-linear shape such that the second band body 160 and the third band body 170 connected to the second hole portions 147a and 147b can be disposed so as to radially change in angle about the pressing portion 120 positioned in the first region 141.

According to the hemostatic device 100 configured as described above, of the plurality of hole portions, the pair of second hole portions 147a and 147b are larger than the widths of the band bodies 160 and 170 and configured in a non-linear shape such that the second band body 160 and the third band body 170 connected to the second hole portions 147a and 147b can be disposed so as to radially change in angle about the pressing portion 120 positioned in the first region 141. Accordingly, in the hemostatic device 100, it is possible to adjust the angle and position of each of the band bodies 160 and 170 with respect to the patient's right hand H1 by moving the band bodies 160 and 170 respectively connected to the second hole portions 147a and 147b about the pressing portion 120 so as to be radially disposed along the respective hole portions 147a and 147b while disposing the pressing member 110 at the first puncture site p1 formed in the patient's right hand H1. As a result, with the hemostatic device 100, each of the band bodies 160 and 170 can be disposed on the patient's right hand H1 such that the movement of the patient's right hand H1 is not restrained, and the hemostatic device 100 can be rather easily mounted onto the patient's right hand H1.

In addition, the second region 142 of the support member 140 has the pair of first hole portions 145a and 145b facing each other with the pressing portion 120 interposed between the pair of first hole portions 145a and 145b and the pair of second hole portions 147a and 147b facing each other with the pressing portion 120 interposed between the pair of second hole portions 147a and 14b at positions different from the first hole portions 145a and 145b. The first hole portions 145a and 145b and the second hole portions 147a and 147b are disposed on a virtual circle along the outer shape of the support member 140. Each of the first band body 150, the second band body 160, and the third band body 170 is disposed in the different hole portion of either the first hole portions 145a and 145b or the second hole portions 147a and 147b.

In the hemostatic device 100 configured as described above, for example, the first band body 150 can be connected to the first hole portion 145a, the second band body 160 can be connected to the second hole portion 147a, and the third band body 170 can be connected to the second hole portion 147b. In addition, the first hole portions 145a and 145b and the second hole portions 147a and 147b are disposed on a virtual circle along the outer shape of the support member 140. Accordingly, the second band body 160 can be disposed so as to radially change in angle about the pressing portion 120 in a state of being connected to the second hole portion 147a different from each of the first hole portions 145a and 145b and the second hole portion 147b. Likewise, the third band body 170 can be disposed so as to radially change in angle about the pressing portion 120 in a state of being connected to the second hole portion 147b different from each of the first hole portions 145a and 145b and the second hole portion 147a. Accordingly, in mounting the hemostatic device 100 onto the patient's right hand H1, an operator can rather easily and finely adjust the position of each of the band bodies 160 and 170 with respect to the right hand H1 with the pressing portion 120 as a fulcrum and can help prevent a positional deviation of the pressing portion 120 from the first puncture site p1 formed in the patient's right hand H1.

In addition, the pair of first hole portions have the one first hole portion 145a and the other first hole portion 145b disposed at positions on the distal side of the patient's right hand H1 or positions on the proximal side of the patient's right hand H1 with the pressing portion 120 interposed between the one first hole portion 145a and the other first hole portion 145b. The pair of second hole portions have the one second hole portion 147a and the other second hole portion 147b disposed with the pressing portion 120 interposed between the one second hole portion 147a and the other second hole portion 147b in a direction intersecting the straight line C connecting the one first hole portion 145a and the other first hole portion 145b. The first band body 150 is connected to the one first hole portion 145a or the other first hole portion 145b. The second band body 160 is connected to the one second hole portion 147a in a slidable state. The third band body 170 is connected to the other second hole portion 147b in a slidable state.

According to the hemostatic device 100 configured as described above, the one second hole portion 147a and the other second hole portion 147b are disposed with the pressing portion 120 interposed between the one second hole portion 147a and the other second hole portion 147b in a direction intersecting the straight line C connecting the distal side position of the patient's right hand H1 and the proximal side position of the patient's right hand H1. Accordingly, as for the hemostatic device 100, when the hemostatic device 100 is mounted onto the patient's right hand H1, it is possible to wind the second band body 160 connected to the one second hole portion 147a in a slidable state and the third band body 170 connected to the other second hole portion 147b in a slidable state along the outer circumference of the patient's right hand H1 while pressing the side surface of the pressing portion 120 against the body surface of the patient's right hand H1. In addition, in winding the second band body 160 and the third band body 170 along the outer circumference of the patient's right hand H1, by sliding each of the band bodies 160 and 170 about the center point R provided in the first region 141 of the support member 140, the position of each of the band bodies 160 and 170 can be rather easily adjusted such that the movement of the patient's right hand H1 is not restrained.

In addition, the respective hole lengths L3 and L4 of the pair of second hole portions 147a and 147b are longer than the respective hole lengths L1 and L2 of the pair of first hole portions 145a and 145b.

According to the hemostatic device 100 configured as described above, the respective hole lengths L3 and L4 of the pair of second hole portions 147a and 147b are longer than the respective hole lengths L1 and L2 of the pair of first hole portions 145a and 145b, and thus the respective slide angles 81 and 82 of the band bodies 160 and 170 respectively connected to the second hole portions 147a and 147b can be increased. Accordingly, in mounting the hemostatic device 100 onto the patient's right hand H1, the position of each of the band bodies 160 and 170 can be rather easily adjusted while preventing a positional deviation of the pressing portion 120 from the first puncture site p1 formed in the patient's right hand H1.

In addition, the first curved region 148a convexly curved toward a side away from the pressing portion 120 disposed on the one surface 140a of the support member 140 is formed at the part of the second region 142 of the support member 140 where the pair of first hole portions 145a and 145b are disposed.

According to the hemostatic device 100 configured as described above, in mounting the hemostatic device 100 onto the patient's hand H, an operator can dispose the first curved region 148a formed near the one first hole portion 145a at a distal side position of the patient's right hand H1 and can dispose the first curved region 148a formed near the other first hole portion 145b at a proximal side position of the patient's right hand H1. By disposing the hemostatic device 100 in this manner, when the patient twists his or her wrist to move the right hand H1 in the up-down or right-left direction with the hemostatic device 100 mounted on the patient's right hand H1, the first curved region 148a helps prevent the peripheral edge portion of the support member 140 from abutting against the patient's right hand H1. As a result, while hemostasis is performed with the hemostatic device 100 at the first puncture site p1 formed in the patient's right hand H1, it is possible to help prevent the patient from feeling discomfort or pain due to the peripheral edge portion of the support member 140 abutting against or biting into the patient's right hand H1.

In addition, the second curved region 148b convexly curved toward the side of the pressing portion 120 disposed on the one surface 140a of the support member 140 is formed at the part of the second region 142 of the support member 140 where the pair of second hole portions 147a and 147b are disposed.

According to the hemostatic device 100 configured as described above, in mounting the hemostatic device 100 onto the patient's hand H, an operator can fix the pressing portion 120 to the patient's right hand H1 such that the second curved region 148b formed on the support member 140 is disposed along a part of the outer circumference of the patient's right hand H1. When the pressing portion 120 inflates with the hemostatic device 100 mounted on the patient's right hand H1 in this manner, the second curved region 148b of the support member 140 presses the pressing portion 120 along a part of the outer circumference of the patient's right hand H1. As a result, the hemostatic device 100 is capable of helping prevent the pressing portion 120 from floating up from the patient's right hand H1. Accordingly, the hemostatic device 100 is capable of effectively applying a compressive force to the first puncture site p1 by the inflatable member 130.

Next, the modification example of the hemostatic device according to the invention will be described. In the description of the modification example, description of members, procedures of hemostatic device use, and so on already described in the description of the embodiment described above will be omitted as appropriate. In addition, content not particularly described in each modification example can be the same as that in the embodiment described above.

### Modification Example

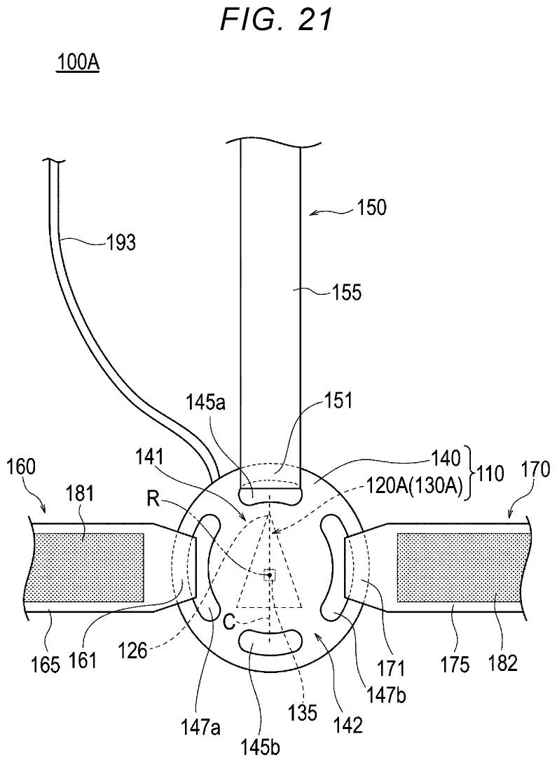

FIG. 21 is an enlarged view of a part of a hemostatic device 100A according to the modification example.

As illustrated in FIG. 21, a pressing portion 120A (inflatable member 130A) of the hemostatic device 100A according to the modification example is configured to gradually decrease in width from the other first hole portion 145b side toward the one first hole portion 145a side.

Specifically, the pressing portion 120A has the shape of an isosceles triangle having a vertex 126 disposed on the one first hole portion 145a side in the plan view illustrated in FIG. 21. Note that the width of the pressing portion 120A is a dimension in a direction orthogonal to the straight line C connecting the one first hole portion 145a and the other first hole portion 145b (right-left direction in FIG. 21).

In addition, in the hemostatic device 100A according to this modification example, the first band body 150 is configured to be connectable to and separable from the one first hole portion 145a and the other first hole portion 145b. The first one end portion 151 of the first band body 150 is provided with a fixing member (for example, a hook-and-loop fastener) capable of holding and releasing the first one end portion 151 in a state of being wound around each of the hole portions 145a and 145b.

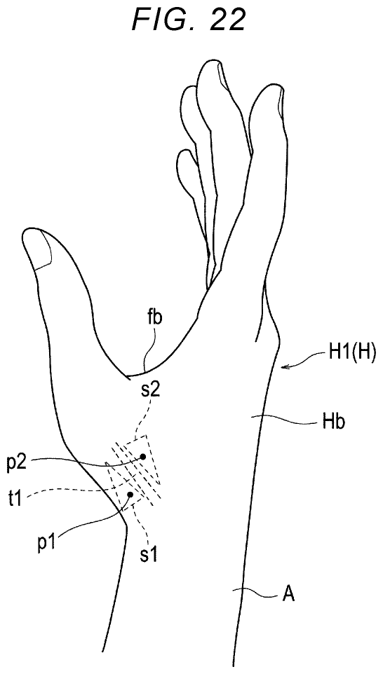

Exemplified in FIG. 22 are a first puncture spot s1 including the first puncture site p1 formed in the patient's right hand H1 and the surrounding portion and a second puncture spot s2 including the second puncture site p2 formed in the patient's right hand H1 and the surrounding portion.

The first puncture spot s1 includes a predetermined range around the snuff box. The first puncture spot s1 has the shape of a substantially isosceles triangle tapering toward the distal side of the patient's right hand H1. In addition, a recessed portion recessed inside the extensor pollicis longus tendon t1 in the patient's right hand H1 when the patient spreads the thumb of the right hand H1 is formed at the first puncture spot s1.

The second puncture spot s2 includes a predetermined range closer to the distal side of the patient's right hand H1 than the snuff box with respect to the extensor pollicis longus tendon t1. The second puncture spot s2 has the shape of a substantially isosceles triangle tapering toward the proximal side of the patient's right hand H1. In addition, a recessed portion recessed inside the extensor pollicis longus tendon t1 in the patient's right hand H1 when the patient spreads the thumb of the right hand H1 is formed at the second puncture spot s2.

In a case where an operator performs hemostasis at the first puncture site p1 formed in the patient's right hand H1 using the hemostatic device 100A, the operator disposes the pressing portion 120A at the first puncture spot s1. In disposing the pressing portion 120A at the first puncture spot s1, the operator disposes the vertex 126 of the pressing portion 120A on the distal side of the patient's right hand H1. By disposing the pressing portion 120A as described above, the operator can dispose the pressing portion 120A so as to overlap the first puncture spot s1. As a result, in the hemostatic device 100A, the pressing portion 120A is capable of effectively applying a compressive force to the first puncture spot s1 including the first puncture site p1 and the surrounding portion. In addition, in the hemostatic device 100A, by disposing the pressing portion 120A at the first puncture spot s1 where a recessed portion recessed inside the patient's right hand H1 when the patient spreads the thumb of the right hand H1 is formed, a positional deviation of the pressing portion 120A from the first puncture site p1 formed in the patient's right hand H1 can be prevented.

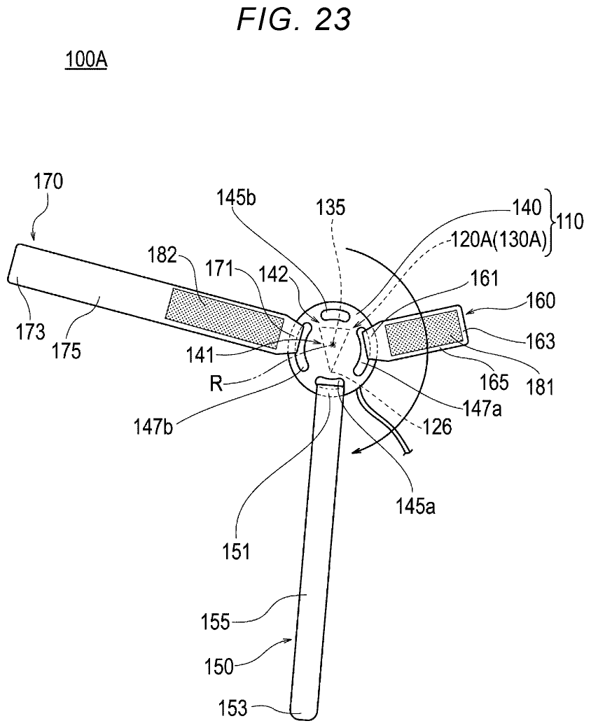

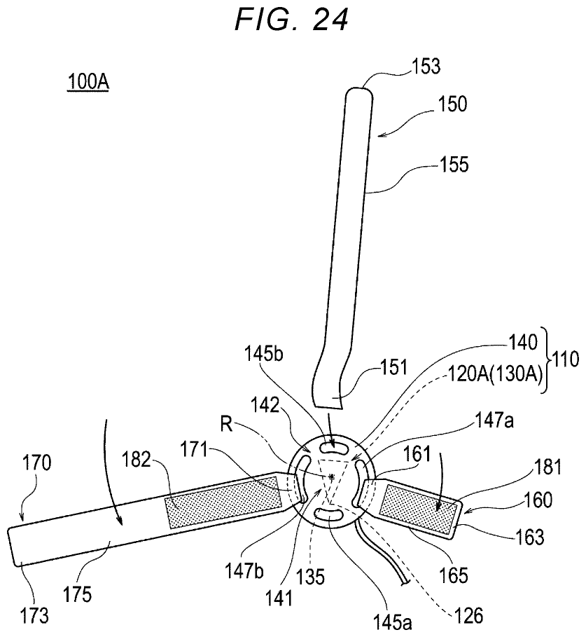

In a case where the operator performs hemostasis at the second puncture site p2 formed in the patient's right hand H1 using the hemostatic device 100A, the operator changes the orientation of the hemostatic device 100A and mounts the hemostatic device 100A onto the patient's right hand H1 as illustrated in FIG. 23. Specifically, the operator reverses the orientation of the hemostatic device 100A in the up-down direction in FIG. 23 such that the vertex 126 of the pressing portion 120A is disposed at the proximal side position of the patient's right hand H1. The operator can dispose the pressing portion 120A so as to overlap the second puncture spot s2 by reversing the orientation of the hemostatic device 100A as described above. As a result, the operator is capable of effectively applying a compressive force to the second puncture spot s2 including the second puncture site p2 and the surrounding portion. In addition, the operator can help prevent a positional deviation of the pressing portion 120A from the second puncture spot s2.

With the orientation of the hemostatic device 100A reversed as described above, the operator removes the first one end portion 151 of the first band body 150 from the one first hole portion 145a and connects the first one end portion 151 of the first band body 150 to the other first hole portion 145b as illustrated in FIG. 24. The operator can dispose the first band body 150 connected to the support member 140 via the other second hole portion 147b on the interdigital portion fb (see FIG. 22) between the thumb and the index finger of the patient's right hand H1. In addition, by radially sliding each of the band bodies 160 and 170 about the center point R provided in the first region 141 of the support member 140, the operator can dispose each of the band bodies 160 and 170 on the patient's right hand H1 such that the movement of the patient's right hand H1 is not restrained. Accordingly, the operator can firmly fix the hemostatic device 100A to the patient's right hand H1.

Note that although an example of applying the hemostatic device 100A to each of the puncture spots s1 and s2 of the patient's right hand H1 has been described in the above example, it is also possible to apply the hemostatic device 100A in the same procedure to each puncture spot including the respective puncture sites p3 and p4 (see FIG. 18) of the patient's left hand H2.

Note that although the pressing portion 120A is configured to gradually decrease in width from the other first hole portion 145b side toward the one first hole portion 145a side in the hemostatic device 100A according to the modification example, the pressing portion 120A may be configured to gradually decrease in width from the one first hole portion 145a side toward the other first hole portion 145b side. In a case where the pressing portion 120A is configured to gradually decrease in width from the one first hole portion 145a side toward the other first hole portion 145b side, when the pressing portion 120A is disposed at the first puncture spot s1, the other first hole portion 145b can be disposed at a position on the peripheral side of the patient's hand H.

In addition, the outer shape of the pressing portion 120A of the hemostatic device 100A according to the modification example is not limited to the isosceles triangle. For example, the shape may be trapezoidal.

Although the hemostatic device according to the disclosure has been described above through the embodiment, the disclosure is not limited to the content described in the specification and can be appropriately changed based on the description of the claims.

Exemplified in the description of the embodiment is a hemostatic device configured such that the second band body and the third band body can be disposed so as to radially change in angle about the pressing portion and the first band body is incapable of radially changing in angle about the pressing portion. However, the hemostatic device may be configured such that at least two of the first band body, the second band body, and the third band body can be disposed so as to radially change in angle about the pressing portion.

Each band body in the description of the embodiment is configured to be slidable about the center point provided in the first region of the support member in a state where the band bodies are respectively connected to the hole portions of the support member. However, the structure for connecting each band body to the support member is not limited to the hole portion provided in the support member. For example, the structure for connecting each band body to the support member can also be configured by a protruding portion provided on one of the support member and each band body and a groove provided in the other of the support member and each band body and holding the protruding portion so as to be slidable.

In the description of the embodiment, the support member is formed in a circular shape in a plan view and each hole portion is disposed on a virtual circle along the outer shape of the support member. However, the outer shape of the support member and the disposition and shape of each hole portion are not particularly limited insofar as at least two of the first band body, the second band body, and the third band body can be configured so as to be capable of being disposed so as to radially change in angle about the pressing portion. For example, in a case where the pressing portion is elliptical, the two band bodies may be configured so as to be capable of being disposed so as to respectively and radially change in angle about different center points provided in the first region.

A hemostatic device for hemostasis at a puncture site formed on the dorsal side of one's hand has been exemplified in the description of the embodiment. However, the hemostatic device can also be used for hemostasis at a puncture site formed at a palmar puncture site. In addition, the disposition of each band body at the time of hemostatic device mounting onto a patient is not limited to the position described by illustration and can be appropriately changed. For example, the first band body can also be disposed in an interdigital portion other than the interdigital portion positioned between the thumb and the index finger. In addition, the hemostatic device may be used for a foot, which has many moving parts such as toes, as in the case of the hand. For example, the hemostatic device may be used for hemostasis at a puncture site in a patient's foot (for example, instep or sole puncture site).

The shape, dimensions, and so on of each portion of the hemostatic device are not particularly limited and can be appropriately changed insofar as the inflatable member can be disposed at a site where bleeding is to be stopped.

The detailed description above describes embodiments of a hemostatic device. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A hemostatic device comprising:
a pressing member configured to compress a puncture site formed in a patient;
a first band body configured to be connectable to the pressing member;
a second band body configured to be connectable to the pressing member;
a third band body configured to be connectable to the pressing member;
the pressing member including a pressing portion configured to compress the puncture site and a support member configured to fix the pressing portion;
the support member including a first region where the pressing portion is disposed and a second region positioned outside the first region;
the first band body, the second band body, and the third band body are configured to be respectively connectable to a plurality of hole portions provided in the second region of the support member; and
at least two of the plurality of hole portions having widths that are larger than a width of the first band body, a width of the second band body, and a width of the third band body, the at least two of the plurality of hole portions being configured in a non-linear shape, and wherein one or more of the first band body, the second band body, and the third band body are configured to be connected to the at least two hole portions and configured to radially change in angle about the pressing portion relative to the first region.

2. The hemostatic device according to claim 1, wherein
the plurality of hole portions include a pair of first hole portions facing each other with the pressing portion interposed between the pair of first hole portions and a pair of second hole portions facing each other with the pressing portion interposed between the pair of second hole portions at positions different from the first hole portions;
the pair of first hole portions and the pair of second hole portions are disposed on a virtual circle along an outer shape of the support member; and
each of the first band body, the second band body, and the third band body is disposed in the different hole portion of either the pair of first hole portions or the pair of second hole portions.

3. The hemostatic device according to claim 2, wherein
the pair of first hole portions include one first hole portion and an other first hole portion configured to be disposed at positions on a distal side of a hand of the patient or positions on a proximal side of the hand of the patient with the pressing portion interposed between the one first hole portion and the other first hole portion;
the pair of second hole portions include one second hole portion and an other second hole portion disposed with the pressing portion interposed between the one second hole portion and the other second hole portion in a direction intersecting a straight line connecting the one first hole portion and the other first hole portion;
the first band body is configured to be connected to the one first hole portion or the other first hole portion;
the second band body is configured to be connected to the one second hole portion in a slidable state; and
the third band body is configured to be connected to the other second hole portion in a slidable state.

4. The hemostatic device according to claim 2, wherein the pressing portion decreases in width from one first hole portion side toward an other first hole portion side between the pair of first hole portions.

5. The hemostatic device according to claim 2, further comprising:
each of the pair of second hole portions is longer in hole length than each of the pair of first hole portions.

6. The hemostatic device according to claim 2, wherein a first curved region convexly curved toward a side away from the pressing portion disposed on one surface of the support member is formed at a part of the second region of the support member where the pair of first hole portions are disposed.

7. The hemostatic device according to claim 2, wherein a second curved region convexly curved toward a side of the pressing portion disposed on one surface of the support member is formed at a part of the second region of the support member where the pair of second hole portions are disposed.

8. The hemostatic device according to claim 1, further comprising:
a plurality of fixing members, the plurality of fixing members including a first fixing member, a second fixing member, a third fixing member, and a fourth fixing member, the first fixing member being disposed on an outer surface of the second body band, the second fixing member being disposed on an outer surface of the third band body, the third fixing member being disposed on an inner surface of the third band body and configured to come into contact with the first fixing member disposed on the outer surface of the second band body, and the fourth fixing member being disposed on an inner surface of the first band body and configured to come into contact with the second fixing member disposed on the outer surface of the third band body.

9. The hemostatic device according to claim 1, wherein the second band body has a slide angle of 1° to 45° about a center point provided in the first region of the second band body, the slide angle of the second band body being defined by a width of the second one end portion of the second band body and a hole length of the one second hole portion, and the third band body has a slide angle of 1° to 45° about a center point provided in the first region of the third band body, the slide angle of the third band body be defined by a width of the third one end portion of the third band body and a hole length of the other second hole portion.

10. The hemostatic device according to claim 1, wherein the pressing portion includes an inflatable member that is connected to the support member via a connection member.

11. A hemostatic device comprising:
an inflatable member configured to receive a fluid;
a support member connected to the inflatable member, the support member including a first region where the inflatable member is disposed and a second region positioned outside the first region;
a first band body configured to be connectable to the pressing member;
a second band body configured to be connectable to the pressing member;
a third band body configured to be connectable to the pressing member;
the first band body, the second band body, and the third band body are configured to be respectively connectable to a plurality of hole portions provided in the second region of the support member; and
wherein one or more of the first band body, the second band body, and the third band body are configured to be connected to the at least two hole portions and configured to radially change in angle about the inflatable portion relative to the first region.

12. The hemostatic device according to claim 11, wherein
the plurality of hole portions include a pair of first hole portions facing each other with the pressing portion interposed between the pair of first hole portions and a pair of second hole portions facing each other with the pressing portion interposed between the pair of second hole portions at positions different from the first hole portions.

13. The hemostatic device according to claim 12, wherein the pair of first hole portions and the pair of second hole portions are disposed on a virtual circle along an outer shape of the support member.

14. The hemostatic device according to claim 12, wherein the pair of first hole portions include one first hole portion and an other first hole portion configured to be disposed at positions on a distal side of a hand of a patient or positions on a proximal side of the hand of the patient with the inflatable member interposed between the one first hole portion and the other first hole portion; and
the pair of second hole portions include one second hole portion and an other second hole portion disposed with the pressing portion interposed between the one second hole portion and the other second hole portion in a direction intersecting a straight line connecting the one first hole portion and the other first hole portion.

15. The hemostatic device according to claim 12, wherein the inflatable portion decreases in width from one first hole portion side toward an other first hole portion side between the pair of first hole portions.

16. The hemostatic device according to claim 12, further comprising:
each of the pair of second hole portions is longer in hole length than each of the pair of first hole portions.

17. The hemostatic device according to claim 12, wherein a first curved region convexly curved toward a side away from the pressing portion disposed on one surface of the support member is formed at a part of the second region of the support member where the pair of first hole portions are disposed.

18. The hemostatic device according to claim 12, wherein a second curved region convexly curved toward a side of the pressing portion disposed on one surface of the support member is formed at a part of the second region of the support member where the pair of second hole portions are disposed.

19. The hemostatic device according to claim 12, further comprising:
a plurality of fixing members, the plurality of fixing members including a first fixing member, a second fixing member, a third fixing member, and a fourth fixing member, the first fixing member being disposed on an outer surface of the second body band, the second fixing member being disposed on an outer surface of the third band body, the third fixing member being disposed on an inner surface of the third band body and configured to come into contact with the first fixing member disposed on the outer surface of the second band body, and the fourth fixing member being disposed on an inner surface of the first band body and configured to come into contact with the second fixing member disposed on the outer surface of the third band body.

20. A method comprising:
wrapping a first band body, a second band body, and a third band body of a hemostatic device along an outer circumference of a puncture site of a living body, the hemostatic device includes a pressing member, the pressing member including a pressing portion configured to compress the puncture site of the living body and a support member configured to fix the pressing portion, the support member including a first region where the pressing portion is disposed and a second region positioned outside the first region, the first band body, the second band body, and the third band body being connected to a plurality of hole portions provided in the second region of the support member, and at least two of the plurality of hole portions having widths that are larger than a width of the first band body, a width of the second band body, and a width of the third band body;
radially changing in angle about the pressing portion relative to the first region, one or more of the first band body, the second band body, and the third band body connected to the at least two hole portions; and
injecting a fluid into of the pressing portion disposed on a surface positioned on a side opposite to a surface of the support member of the pressing member to apply a compressive force to the puncture site of the living body.
